(19) United States
(12) Patent Application Publication
(10) Pub. No.: US 2020/0094071 A1
(43) Pub. Date: Mar. 26, 2020

(54) Systems and Methods for Treating Androgenetic Alopecia

(71) Applicant: Applied Biology, Inc., Irvine, CA (US)

(72) Inventor: Ofer A. Goren, Irvine, CA (US)

(21) Appl. No.: 16/582,380

(22) Filed: Sep. 25, 2019

Related U.S. Application Data
(60) Provisional application No. 62/736,675, filed on Sep. 26, 2018.

(51) Int. Cl. A61N 5/06; A61K 31/506; A61P 17/14

(52) U.S. Cl. CPC A61N 5/0617; A61N 5/062; A61N 2005/0663; A61P 17/14; A61N 5/0616; A61K 31/506

(57) ABSTRACT

Embodiments of the system include a therapeutic device configured to generate electromagnetic radiation (EMR) for treatment of androgenetic alopecia. Embodiments of the therapeutic device can be configured to generate EMR having a wavelength within a therapeutic range of wavelengths, the EMR being able to stimulate opsin receptors located in keratinocytes and/or hair follicles so as to cause the stimulated opsin receptors to grow hair and/or promote hair follicle stem cell differentiation and/or proliferation. The therapeutic device can be a unit that is attachable to and communication with a mobile electronic device (e.g., a mobile phone, a tablet, etc.). Data related to the therapy session can be collected and analyzed by the mobile electronic device to provide diagnostic results of the therapy session.

### CROSS-REFERENCE TO RELATED APPLICATIONS

This application is related to and claims the benefit of U.S. Provisional Application 62/736,675 filed on Sep. 26, 2018, the entire contents of which is incorporated herein by reference.

### FIELD OF THE INVENTION

Embodiments of the invention relate to systems and methods for treating alopecia using electromagnetic radiation therapy.

### BACKGROUND OF THE INVENTION

Conventional methods for treating alopecia are limited in that they fail to provide a means to adjust therapy sessions based on real time information collected about the therapy session.

### SUMMARY OF THE INVENTION

Embodiments of the system include a therapeutic device configured to generate electromagnetic radiation (EMR) for treatment of androgenetic alopecia. Embodiments of the therapeutic device can be configured to generate EMR having a wavelength within a therapeutic range of wavelengths, which can include wavelengths within 400 nm to 495 nm and/or 630 nm to 670 nm. The EMR within a therapeutic range has a wavelength that can stimulate opsin receptors located in keratinocytes and/or hair follicles so as to cause the stimulated opsin receptors to grow hair. The wavelengths within the therapeutic range of can further promote hair follicle stem cell differentiation and/or proliferation. The therapeutic device can be a unit that is attachable to and communication with a mobile electronic device (e.g., a mobile phone, a tablet, etc.). Data related to the therapy session can be collected and analyzed by the mobile electronic device to provide diagnostic results of the therapy session.

Further features, aspects, objects, advantages, and possible applications of the present invention will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present invention will be more apparent from the following more particular description thereof, presented in conjunction with the following drawings. Like reference numbers used in the drawings may identify like components.

FIG. 1 shows an embodiment of the system with an embodiment of the therapeutic device attached to an embodiment of a mobile electronic device.

FIG. 2 shows an embodiment of the system being used to provide an exemplary treatment for alopecia.

FIG. 3 shows an embodiment of the therapeutic device filtering electromagnetic radiation to wavelengths within the therapeutic range.

FIG. 4 shows an exemplary communications network that can be used for an embodiment of the system.

### DETAILED DESCRIPTION OF THE INVENTION

The following description is of exemplary embodiments that are presently contemplated for carrying out the present invention. This description is not to be taken in a limiting sense, but is made merely for the purpose of describing the general principles and features of the present invention. The scope of the present invention is not limited by this description.

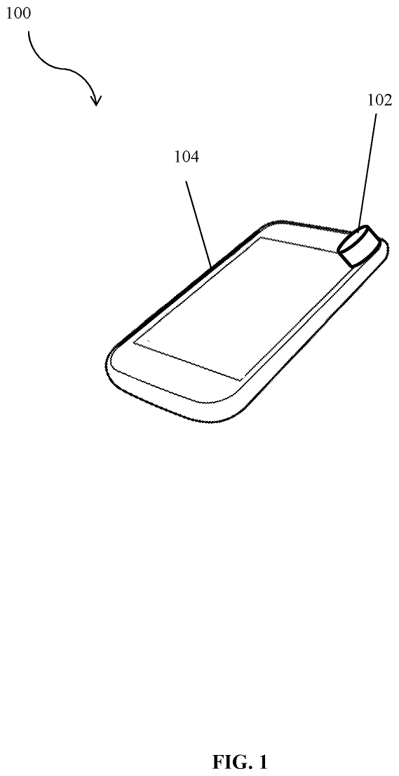

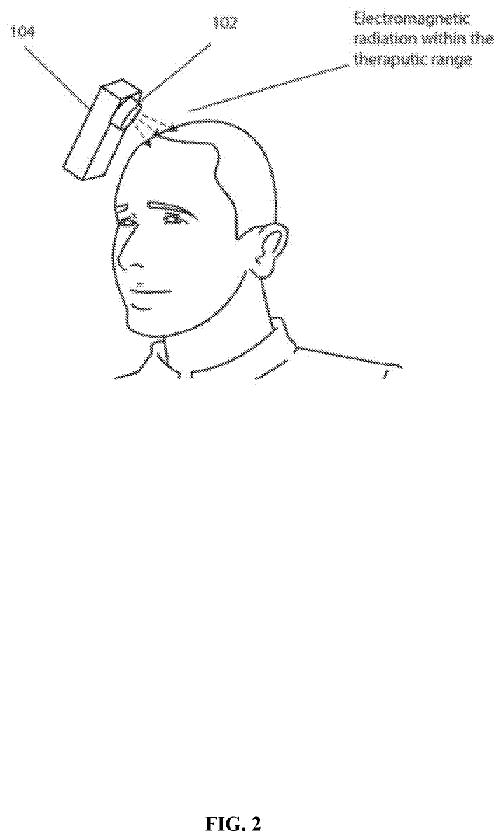

Referring to FIGS. 1-2, embodiments of the system 100 can include a therapeutic device 102 configured to generate electromagnetic radiation (EMR) for treatment of alopecia. This can include treatment of androgenetic alopecia. Treatment can be defined as preventing alopecia, reducing the effects of alopecia, and/or reversing the effects of alopecia. Embodiments of the therapeutic device 102 can be configured to generate EMR having a wavelength within a therapeutic range of wavelengths. It is contemplated for the therapeutic range to be within 400 nm to 495 nm (i.e., blue light therapeutic range) and/or 630 nm to 670 nm (i.e., red light therapeutic range). Thus, the EMR can include at least one wavelength within the range from 400 nm to 495 nm and/or within the range from 630 nm to 670 nm. The EMR within a therapeutic range has a wavelength that can stimulate opsin receptors located in keratinocytes and/or hair follicles so as to cause the stimulated opsin receptors to grow hair. The EMR within the therapeutic range of can further promote hair follicle stem cell differentiation (e.g., via photobiomodulation) and/or proliferation (e.g., cellular activity modulation). Thus, while the blue light therapeutic range and the red light therapeutic range are discussed herein, it will be appreciated that other therapeutic wavelengths can be used—i.e., any EMR having a wavelength that can stimulate opsin receptors located in keratinocytes and/or hair follicles so as to cause the stimulated opsin receptors to grow hair and/or promote hair follicle stem cell differentiation and/or proliferation can be used.

The therapeutic device 102 can be a unit that is attachable to a mobile electronic device 104 (e.g., a mobile phone, a tablet, etc.). The therapeutic device 102 can be permanently attached to the mobile electronic device 104 via an adhesive, fasteners, etc. The therapeutic device 102 can be temporarily attached to the mobile electronic device 104 via a suction mechanism, an interference fit, a clip arrangement, etc. As explained herein, the therapeutic device 102 can be configured to generate the EMR via an illuminator 106 so that the generated EMR is within the therapeutic range. In addition, or in the alternative, the therapeutic device 102 can be configured to filter EMR generated by an illuminator 106 of the mobile electronic device 104 so as to output EMR being within the therapeutic range.

In some embodiments, the therapeutic device 102 can include a filter 108 (e.g., a band-pass filter) configured to filter EMR being generated by an illuminator 106. The illuminator 106 can be a light source configured to generate EMR. The filter 108 can be attached to the illuminator 106 and pass EMR that is within the therapeutic range. For example, the illuminator 106 can be a light source of the mobile electronic device 104 (e.g., the flashlight illuminator of a smartphone). The filter 108 can be configured to attach to a portion of the mobile electronic device 104 so as to cause the filter 108 to be positioned over the illuminator 106. A user can activate the illuminator 106 of the mobile electronic device 104 to cause the EMR to pass through the filter 108 so that the EMR radiating from the filter 108 is within the therapeutic range.

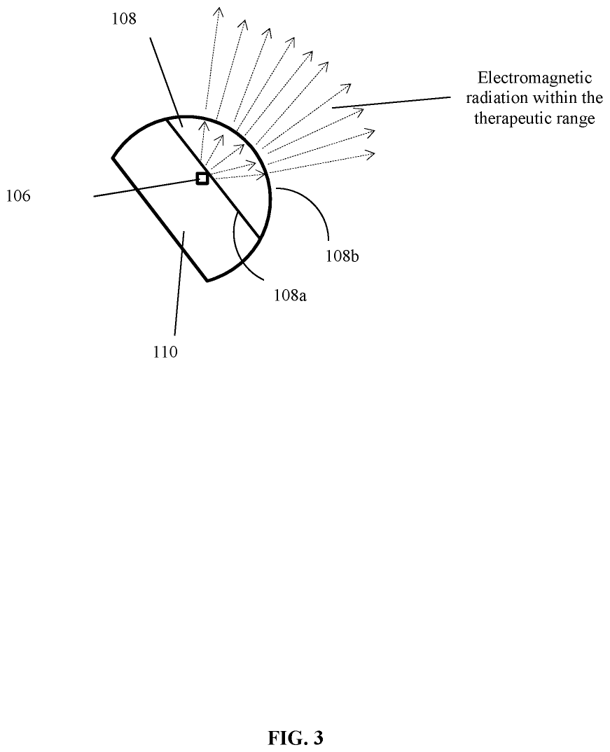

Referring to FIG. 3, as a non-limiting example, the therapeutic device 102 can include a bezel 110 configured to hold a dichroic mirror as the filter 108. The filter 108 can have a filter first side 108a and a filter second side 108b. The bezel 110 can be configured to attach to a portion of the mobile electronic device 104 so as to position the filter 108 over the illuminator 106 of the mobile electronic device 104. This can include causing the filter first side 108a to be adjacent the illuminator 106 and the filter second side 108b subtending the illuminator 106. When the illuminator 106 is activated, EMR (e.g., white light) is caused to be incident on the filter first side 108a and exits the filter second side 108b as EMR having a wavelength within the therapeutic range. As another example, the filter 108 can include at least one thin film, diffraction grating, etc. that can allow for wavelength tuning. Thus, the wavelength of the EMR exiting the filter second side 108b can be adjusted. This can be done to narrow the wavelength range, broaden the wavelength range, shift the wavelength range, etc. Other optical elements, such as lenses, prisms, collimators, attenuators, amplifiers, beam splitters, photo-array sensors, etc. can be used to control aspects of the EMR, adjust the intensity of the EMR, cause the EMR to follow desired paths, generate EMR pulses, adjust the pulse duration and pulse frequency, etc.

As noted herein, some embodiments of the therapeutic device 102 include the illuminator 106. As a non-limiting example, the therapeutic device 102 can include a bezel 110 configured to hold the illuminator 106. The illuminator 106 can be a light emitting diode (LED) or other light generator configured to emit EMR having a wavelength within the therapeutic range. The therapeutic device 102 can be attached to the mobile electronic device 104 as described above. A user can activate the illuminator 106 to cause the illuminator 106 to generate the EMR having a wavelength within the therapeutic range. Some embodiments can include a plurality of illuminators 106. For example, the bezel 110 can hold a first illuminator 106 configured to generate EMR within the blue light therapeutic range, a second illuminator 106 configured to generate EMR within the red light therapeutic range, etc. Again, other optical elements, such as lenses, prisms, collimators, attenuators, amplifiers, beam splitters, photo-array sensors, etc. can be used to control aspects of the EMR, adjust the intensity of the EMR, cause the EMR to follow desired paths, generate EMR pulses, adjust the pulse duration and pulse frequency, etc.

In some embodiments, the therapeutic device 102 can include a filter 108 and its own illuminator 106. The filter 108 can be tunable to allow a user to adjust the wavelength of EMR being generated by the illuminator 106.

In some embodiments, the bezel 110 portion of the therapeutic device 102 can have bristles or other protrusions (e.g., nubs, protuberances, etc.). The bristles can be used to massage the skin upon which the EMR is incident to facilitate penetration of the EMR into the skin.

Embodiments of the therapeutic device 102 can include a power source. The power source can be a power outlet (e.g., power cord socket, USB port, etc.) configured to connect the therapeutic device 102 to the mobile electronic device 104.

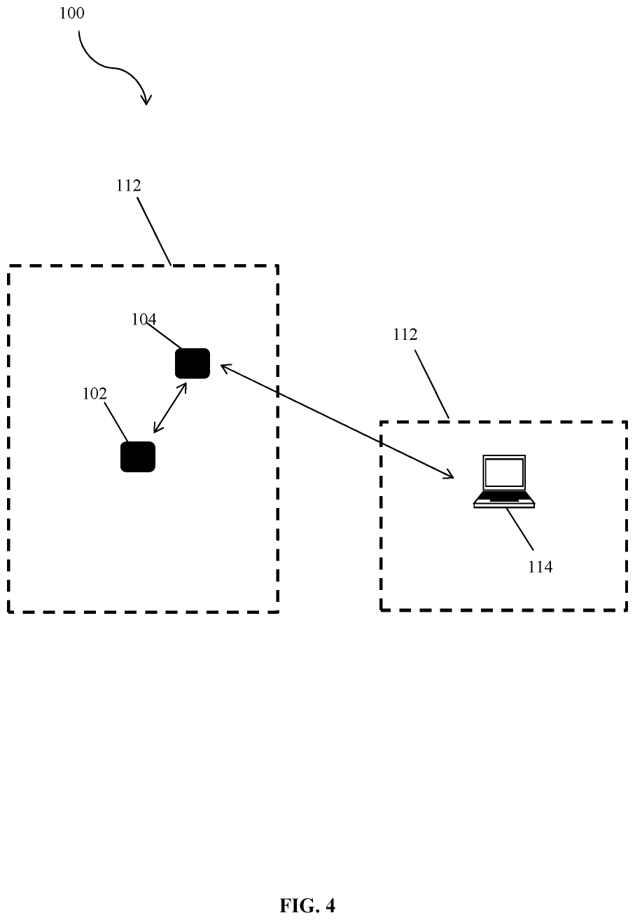

Referring to FIG. 4, in some embodiments, the therapeutic device 102 can be part of or in connection with a communications network 112. For example, the therapeutic device 102 can include switches, transmitters, transceivers, routers, gateways, etc. to facilitate communications via a communication protocol that facilitates controlled and coordinated signal transmission and processing. The communication links can be established by communication protocols that allow a therapeutic device 102 to form the communications network 112 with another apparatus 114 (e.g., the mobile electronic device). For instance, the therapeutic device 102 can be configured to communicate with the mobile electronic device 104 and to facilitate data transmissions to and from or other nodes 114 (e.g., other third party computer devices connected to the communications network) within or between discrete communication networks. The communications network 112 can be a long range wired or a wireless network, such as an Ethernet, telephone, Wi-Fi, Bluetooth, wireless protocol, cellular, satellite network, cloud computing network, etc. Embodiments of the communications network 112 can be configured as a predetermined network topology. This can include a mesh network topology, a point-to-point network topology, a ring (or peer-to-peer) network topology, a star (point-to-multiple) network topology, or any combination thereof.

With embodiments of the therapeutic device being able to be part of a communications network 112, the system 100 can be configured so that the therapeutic device 102 can communicate and transfer data wirelessly with the mobile electronic device 104 and/or other third party computer devices 114 connected to the communications network 112.

In some embodiments, the therapeutic device 102 can include a processor in operative association with a non-volatile, non-transitory memory. The processor can control components (e.g., the optical elements) of the therapeutic device 102 and facilitate communications and data transfer between the therapeutic device 102, the mobile electronic device 104, and/or third party computer devices 114.

In addition, therapeutic device 102 can have an application programming interface (API) and/or other interfaces configured to facilitate a computer device (e.g., the mobile electronic device 104 and/or third party computer devices 114) that is in communication with the therapeutic device 102 executing commands and controlling aspects of the therapeutic device 102. For example, the mobile electronic device 104 can be programmed to generate a user interface configured to facilitate control of and display of various operational aspects of the therapeutic device 102 (e.g., EMR wavelength, EMR intensity, EMR pulse generation, EMR pulse width, EMR pulse frequency, EMR pulse duration, etc.). The user interface can include interactive elements to allow a user to set parameters of the therapy session. These parameters can include the operational aspects of the therapeutic device 102, the duration of the therapy session, the illumination power of the EMR generated during the therapy session, etc. The user interface can also include interactive elements to allow a user to derive a program of therapy sessions, the program being a plurality of therapy sessions. Any one or combination of therapy sessions can be the same as or different from another therapy session in the program. The user interface can also provide a user with a schedule (e.g., a calendar) corresponding to when the therapy sessions are to be performed. Some embodiments can include generating messages (e.g., text messages, emails, etc.) or alerts (alarms, notifications, etc.) to remind the user to conduct a therapy session in accordance with the program. The user interface can also provide image processing, diagnostic features, etc. It is contemplated for the user interface to be implemented via application software (an “app”) installed on the mobile electronic device.

The application software can allow a user to develop his or her own therapy sessions and/or programs. In addition, or in the alternative, the application software can develop the therapy sessions and/or programs based on physiological and biological information about the user and the user's skin or hair condition. This can include taking into account times of the day in which the therapy session are more effective due to cell activity. For example, human cells may be more active during daylight hours so a circadian clock or a circadian oscillator can be used to derive the therapy sessions and/or develop the schedules for the therapy sessions that make up the programs.

It is contemplated for the mobile electronic device 104 to be able to monitor usage, track and record therapeutic sessions, track and record EMR wavelengths and intensities used, record images and/or video of tissue being subjected to the therapy, provide diagnostic results, etc. This can be achieved by the camera function of the mobile electronic device 104 capturing images and/or video of the tissue. These images and/or video can be processed using object recognition, signal processing functions (e.g., Fourier transforms, Gabor transforms, etc.), etc. to generate diagnostics about the treatment. The processor and optical elements (e.g., the photo-array sensors) of the therapeutic device 102 can be used to track the EMR wavelengths, the EMR intensities, the time duration of therapeutic sessions, etc., which can be transmitted to the mobile electronic device 104 for further processing. These data can be used to monitor the therapy session, display progress of the treatment, provide recommendations for therapy adjustment, etc.

Because the therapeutic device 102 and/or the mobile electronic device 104 can be in communication with the communications network 112, the therapeutic device 102 and/or the mobile electronic device 104 can be placed into communication with third party computer devices 114. Embodiments of the third party computer devices 114 can include a server, a mainframe computer, a desk top computer, a laptop computer, another tablet, another smartphone, etc. These third party computer devices 114 can also be programmed to generate a user interface configured to facilitate control of and display of various operational aspects of the therapeutic device 102 (e.g., EMR wavelength, EMR intensity, EMR pulse generation, EMR pulse width, EMR pulse frequency, EMR pulse duration, etc.). The user interface can include interactive elements to allow a third party user to set parameters of the therapy session. These parameters can include the operational aspects of the therapeutic device 102, the duration of the therapy session, the illumination power of the EMR generated during the therapy session, etc. The user interface can also include interactive elements to allow a third party user to derive a program of therapy sessions, the program being a plurality of therapy sessions. Any one or combination of therapy sessions can be the same as or different from another therapy session in the program. Again, the application software can develop the therapy sessions and/or programs based on physiological and biological information about the user and the user's skin or hair condition. This can include taking into account times of the day in which the therapy session are more effective due to cell activity. For example, human cells may be more active during daylight hours so a circadian clock or a circadian oscillator can be used to derive the therapy sessions and/or develop the schedules for the therapy sessions that make up the programs.

It is contemplated for the third party computer device 114 to be operated by a third party who may be a specialist on the treatment of alopecia (e.g., a doctor, dermatologist, cosmetic consultant, etc.). The diagnostics of the therapy session can be transmitted to the third party computer device 114. The specialist can provide recommendations to the patient (e.g., the one receiving the treatment and the user of the mobile electronic device) via the user interface of the third party computer device 114, which can be transmitted to the user interface of the mobile electronic device 104. The recommendations can include adjustments to the therapy session (e.g., adjustments in EMR wavelength, EMR intensity, EMR pulse duration, frequency of therapy sessions, etc.). In addition, or in the alternative, the system 100 can be configured such that the third party computer device 114 transmits instructions to the mobile electronic device 104 that automatically make adjustments to the therapy session, which are then transmitted to the therapeutic device 102. The recommendations can also include suggesting an appointment with a specialist. As will be explained, the methods of treatment can be augmented via use of topological or oral compositions containing an agonist configured to activate the opsin receptor and/or promote hair follicle stem cell differentiation and/or proliferation. An example of such a topological or oral composition can be minoxodil. Thus, the recommendations can include suggested uses of topological or oral compositions.

While embodiments demonstrate use of EMR therapy for the treatment of alopecia, the EMR therapy can also be used to treat acne. For example, EMR within the therapeutic range can be used to reduce or eliminate follicular inflammation. Reducing or eliminating follicular inflammation can be done to treat acne. Thus, embodiments of the therapeutic device can be used to generate EMR having a wavelength within a therapeutic range of wavelengths so that the EMR is make to be incident on follicles that are inflamed.

As noted above, the treatment method involving EMR therapy can be augmented with the use of a topological or oral composition that includes an agonist configured to activate the opsin receptor and/or promote hair follicle stem cell differentiation and/or proliferation. In addition, the EMR therapy can be used to improve or enhance treatment of alopecia via the application topological or oral compositions. For example, follicular inflammation can reduce the efficacy of a topological or oral composition used for the treatment of alopecia. Embodiments of the EMR therapy can be used to reduce or eliminate follicular inflammation so as to improve or enhance the efficacy of the topological or oral composition being used for the treatment of alopecia. Thus, use of embodiments of EMR therapy in combination with a topological or oral composition configured to treat alopecia can be superior to treatment that comprises only the use of the topological or oral composition.

An exemplary implementation can involve attaching an embodiment of the therapeutic device 102 to a portion of a mobile electronic device 104. The therapeutic device 102 can include an illuminator 106 configured to generate EMR having a wavelength within the therapeutic range. In addition, or in the alternative, the therapeutic device 102 can have a filter 108 configured to filter EMR generated from an illuminator 106 of the mobile electronic device 104 so as to generate the EMR having a wavelength within the therapeutic range. The therapeutic device 102 can be placed adjacent tissue exhibiting the effects of alopecia. For example, the therapeutic device 102 can be placed adjacent a portion of the scalp of a person, the portion of the scalp experiencing androgenetic alopecia. The therapeutic device 102 can be caused to generate the EMR having a wavelength within the therapeutic range so that the EMR having a wavelength within the therapeutic range is incident upon the portion of the scalp. This can be done to provide an EMR therapy session to the portion of the scalp, the EMR therapy session being defined as treating alopecia by stimulating opsin receptors located in keratinocytes and/or hair follicles in the portion of the scalp so as to cause the stimulated opsin receptors to grow hair and/or promote hair follicle stem cell differentiation and/or proliferation.

The wavelength, intensity, and other characteristics of the EMR having a wavelength within the therapeutic range can be adjusted. This can be achieved via the user interface of the mobile electronic device 104. The user interface can be used to control operational aspects of the therapeutic device 102 so as to adjust the therapy session. Images and/or video of the portion of the scalp, along with other data (EMR wavelengths, EMR intensities, the time duration of therapeutic sessions, etc.) can be recorded by the mobile electronic device 104. These data can also be transmitted to a third party computer device 114 for additional processing. These data can be used to monitor the therapy session, display progress of the treatment, provide recommendations for therapy adjustment, etc., which can be done by the mobile electronic device 104 and/or the third party computer device 114.

In some embodiments, the EMR therapy session can be used to treat acne. For example, the therapeutic device 102 can be placed adjacent a portion of the skin of a person, the portion of the skin experiencing acne due to follicular inflammation. The therapeutic device 102 can be caused to generate the EMR having a wavelength within the therapeutic range so that the EMR having a wavelength within the therapeutic range is incident upon the portion of the skin. This can be done to provide an EMR therapy session to the portion of the skin, the EMR therapy session being defined as treating acne by reducing or eliminating follicular inflammation.

In some embodiments, the portion of the skin used to provide an EMR therapy session for treating acne can be the same portion of the skin used to provide an EMR therapy session for treating alopecia.

The alopecia treatment via an EMR therapy session can be augmented by application of a topological or oral composition containing an agonist configured to activate the opsin receptor and/or promote hair follicle stem cell differentiation and/or proliferation. In addition, or in the alternative, treating alopecia using a topological or oral composition containing an agonist configured to activate the opsin receptor and/or promote hair follicle stem cell differentiation and/or proliferation can be augmented by providing an EMR therapy session for treating alopecia and/or providing an EMR therapy session for treating acne.

It should be understood that modifications to the embodiments disclosed herein can be made to meet a particular set of design criteria. For instance, the number of or configuration of therapeutic devices 102, illuminators 106, filters 108, mobile electronic devices 104, third party devices 114, communications networks 112, user interfaces, and/or other components or operating parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

Therefore, it is the intent to cover all such modifications and alternative embodiments as may come within the true scope of this invention, which is to be given the full breadth thereof. Additionally, the disclosure of a range of values is a disclosure of every numerical value within that range, including the end points. Thus, while certain exemplary embodiments of apparatuses and methods of making and using the same have been discussed and illustrated herein, it is to be distinctly understood that the invention is not limited thereto but may be otherwise variously embodied and practiced within the scope of the following claims.

It should also be appreciated that some components, features, and/or configurations may be described in connection with only one particular embodiment, but these same components, features, and/or configurations can be applied or used with many other embodiments and should be considered applicable to the other embodiments, unless stated otherwise or unless such a component, feature, and/or configuration is technically impossible to use with the other embodiment. Thus, the components, features, and/or configurations of the various embodiments can be combined together in any manner and such combinations are expressly contemplated and disclosed by this statement. Thus, while certain exemplary embodiments of the system have been shown and described above, it is to be distinctly understood that the invention is not limited thereto but may be otherwise variously embodied and practiced within the scope of the following claims.

What is claimed is:

1. A therapeutic device, comprising:
at least one of:
an illuminator configured to generate electromagnetic radiation (EMR) within a therapeutic range of wavelengths; and
a filter configured to filter EMR being emitted from an illuminator so that EMR within the therapeutic range of wavelengths is passed through the filter;
wherein the EMR within the therapeutic range of wavelengths has a wavelength that at least one of:
stimulates opsin receptors located in keratinocytes and/or hair follicles so as to cause the stimulated opsin receptors to grow hair;
promotes hair follicle stem cell differentiation and/or proliferation; and
reduces follicle inflammation;
wherein the therapeutic device comprises a processor and a transceiver, the therapeutic device being in communication with a mobile electronic device;
wherein the therapeutic device is configured to provide a therapy session for at least one of:
treatment of alopecia;
treatment of acne; and
improve efficacy of a topological or oral composition being used to treat alopecia.

2. The therapeutic device recited in claim 1, further comprising application software configured to cause the therapeutic device to collect data related to the therapy session.

3. The therapeutic device recited in claim 2, wherein the application software transmits the data to the mobile electronic device.

4. The therapeutic device recited in claim 3, wherein the application software causes the mobile electronic device to provide diagnostic results of the therapy session.

5. The therapeutic device recited in claim 3, wherein the application software causes the mobile electronic device to generate a user interface configured to provide a means for a user to control operational aspects of the therapeutic device via the mobile electronic device.

6. The therapeutic device recited in claim 5, wherein the operational aspects of the therapeutic device comprise EMR wavelength, EMR intensity, EMR pulse generation, EMR pulse width, EMR pulse frequency, EMR pulse, duration of the therapy session, and/or illumination power of the EMR generated during the therapy session.

7. The therapeutic device recited in claim 4, wherein the application software causes the therapeutic device and/or the mobile electronic device to transmit the data and/or the diagnostic results to a third party computer.

8. The therapeutic device recited in claim 7, wherein a user of the third party computer transmits recommendations to the mobile electronic device.

9. The therapeutic device recited in claim 7, wherein a user of the third party computer controls operational aspects of the therapeutic device via the third party computer.

10. The therapeutic device recited in claim 1, wherein the therapeutic range comprises EMR having a wavelength within a range from 400 nm to 495 nm and/or within the range from 630 nm to 670 nm.

11. The therapeutic device recited in claim 1, wherein the therapeutic device comprises a first illuminator configured to generate EMR having a wavelength within a range from 400 nm to 495 nm and a second illuminator configured to generate EMR having a wavelength within a range from 630 nm to 670 nm.

12. The therapeutic device recited in claim 1, wherein the alopecia is androgenetic alopecia.

13. A method for treating alopecia, the method comprising:
generating electromagnetic radiation (EMR) within a therapeutic range of wavelengths that will at least one of:
stimulate opsin receptors located in keratinocytes and/or hair follicles so as to cause the stimulated opsin receptors to grow hair;
promotes hair follicle stem cell differentiation and/or proliferation; and
reduce follicle inflammation;
providing a therapy session for at least one of:
treatment of alopecia;
treatment of acne; and
improve efficacy of a topological or oral composition being used to treat alopecia;
collecting data related to the therapy session and transmitting the data to a mobile electronic device; and
generating a diagnostic result and adjusting wavelength and/or intensity of the EMR based on the diagnostic result.

14. The method for treating alopecia recited in claim 13, further comprising controlling aspects of the therapy session via the electronic mobile device, the aspects comprising EMR wavelength, EMR intensity, EMR pulse generation, EMR pulse width, EMR pulse frequency, EMR pulse, duration of the therapy session, and/or illumination power of the EMR generated during the therapy session.

15. The method for treating alopecia recited in claim 13, further comprising transmitting the data and/or the diagnostic result to a third party computer, the aspects comprising EMR wavelength, EMR intensity, EMR pulse generation, EMR pulse width, EMR pulse frequency, EMR pulse, duration of the therapy session, and/or illumination power of the EMR generated during the therapy session.

16. The method for treating alopecia recited in claim 15, further comprising controlling aspects of the therapy session via the third party computer.

17. The method for treating alopecia recited in claim 13, wherein the therapeutic range comprises EMR having a wavelength within a range from 400 nm to 495 nm and/or within the range from 630 nm to 670 nm.

18. The method for treating alopecia recited in claim 13, further comprising attaching a therapeutic device to the mobile electronic device, the therapeutic device being configured to generate the EMR within the therapeutic range.

19. The method for treating alopecia recited in claim 18, further comprising placing the therapeutic device adjacent tissue of a person experiencing effects of alopecia and/or acne.

20. The method for treating alopecia recited in claim 19, further comprising activating an illuminator of the mobile electronic device and/or an illuminator of the therapeutic device via a user interface of the mobile electronic device, the activation generating the EMR within the therapeutic range of wavelengths.

21. The method for treating alopecia recited in claim 20, further comprising filtering the EMR generated by the illuminator.

22. The method for treating alopecia recited in claim 20, wherein the illuminator comprises a plurality of illuminators, each illuminator configured to generate a band of EMR that is within the therapeutic range of EMR.

23. The method for treating alopecia recited in claim 13, wherein the alopecia is androgenetic alopecia.

24. A method for treating alopecia, the method comprising:
application of minoxidil orally or topically; and
applying electromagnetic radiation (EMR) having a wavelength that activates an opsin receptor and/or promotes hair follicle stem cell differentiation and/or proliferation; and.

25. The method recited in claim 24, wherein activating the opsin receptor via the EMR stimulates the opsin receptor to promote hair growth and/or to reduce or eliminate follicle inflammation.

26. The method recited in claim 24, wherein activating the opsin receptor via the EMR stimulates the opsin receptor to promote hair growth and/or to reduce or eliminate follicle inflammation to improve or enhance the efficacy of minoxidil.

27. The method recited in claim 24, wherein the wavelength of the EMR is within a range from 400 nm to 495 nm.

28. A therapeutic device, comprising:
a mobile electronic device comprising an illuminator configured to generate electromagnetic radiation (EMR);
a filter configured to filter EMR being emitted from the illuminator so that EMR within a therapeutic range of wavelengths is passed through the filter, wherein the EMR within the therapeutic range of wavelengths has a wavelength that at least one of:
stimulates opsin receptors located in keratinocytes and/or hair follicles so as to cause the stimulated opsin receptors to grow hair;
promotes hair follicle stem cell differentiation and/or proliferation; and
reduces follicle inflammation;
wherein the therapeutic device comprises a processor and a transceiver, the therapeutic device being in communication with the mobile electronic device;
wherein the therapeutic device is configured to provide a therapy session for at least one of:
treatment of alopecia;
treatment of acne; and
improve efficacy of a topological or oral composition being used to treat alopecia.

29. A therapeutic device, comprising:
an illuminator configured to generate electromagnetic radiation (EMR), the illuminator comprising a filter configured to filter EMR being emitted from the illuminator so that EMR within a therapeutic range of wavelengths is passed through the filter, wherein the EMR within the therapeutic range of wavelengths has a wavelength that at least one of:
stimulates opsin receptors located in keratinocytes and/or hair follicles so as to cause the stimulated opsin receptors to grow hair;
promotes hair follicle stem cell differentiation and/or proliferation; and
reduces follicle inflammation;
wherein the illuminator is configured to be an attachment to a mobile electronic device;
wherein the therapeutic device comprises a processor and a transceiver, the therapeutic device being in communication with the mobile electronic device;
wherein the therapeutic device is configured to provide a therapy session for at least one of:
treatment of alopecia;
treatment of acne; and
improve efficacy of a topological or oral composition being used to treat alopecia.
